(19) United States
(12) Patent Application Publication
(10) Pub. No.: US 2005/0100983 A1
(43) Pub. Date: May 12, 2005

(54) Cell-free methods for identifying compounds that affect toll-like receptor 9 (TLR9) signaling

(75) Inventors: Stefan Bauer, Muenchen (DE); Grayson Lipford, Watertown, MA (US); Hermann Wagner, Eching (DE); Mark Rutz, Muenchen (DE)

Correspondence Address: WOLF GREENFIELD & SACKS, PC;FEDERAL RESERVE PLAZA, 600 ATLANTIC AVENUE, BOSTON, MA, 02210-2211, US

(73) Assignees: Coley Pharmaceutical GmbH, Langenfeld (DE); Coley Pharmaceutical Group, Inc., Wellesley, MA (US); Technische Universitat Munchen, Muenchen (DE)

(21) Appl. No.: 10/982,193

(22) Filed: Nov. 5, 2004

Related U.S. Application Data
(60) Provisional application No. 60/517,804, filed on Nov. 6, 2003.

(51) Int. Cl. C12P 21/06; C07K 14/705

(52) U.S. Cl. 435/68.1; 530/350

(57) ABSTRACT

The invention is directed to methods for screening for a compound that affects interaction between a Toll-like receptor (TLR) and a ligand for the TLR. The methods involve direct measurement of interaction using, for example, surface plasmon resonance (SPR), particularly under conditions of pH that mimic those of the TLR in vivo. Compounds identified using the methods of the invention may be useful in the development of agents useful in the treatment of conditions characterized by undesirable immune activation, e.g., autoimmunity, inflammation, allergy, asthma, and transplantation.

### RELATED APPLICATION

This application claims benefit under 35 U.S.C. 119 of U.S. Provisional Patent Application Ser. No. 60/517,804, filed Nov. 6, 2003, the entire content of which is incorporated by reference herein.

### FIELD OF THE INVENTION

The invention relates to methods of screening for compounds that may affect immune activation. More specifically, the disclosed methods are useful for identifying compounds that affect interaction between Toll-like receptors and their ligands.

### BACKGROUND OF THE INVENTION

Toll receptors are transmembranal proteins which are evolutionarily conserved between insects and vertebrates. Rock F L et al. (1998) Proc. Natl. Acad. Sci. U.S.A. 95:588-593. They are structurally defined by leucine-rich repeats (LRRs) in their extracellular domain and a cytoplasmic signaling Toll/Interleukin-1 Receptor (TIR) domain. Gay N J et al. (1991) Nature 351:355-356. In drosophila, Toll was first identified as an essential molecule for dorsal-ventral patterning of the embryo and subsequently as a key molecule for the antifungal immune response in the adult. Anderson K V et al. (1985) Cell 42:791-798; Lemaitre B et al. (1996) Cell 86:973-983.

A homologous family of Toll receptors, termed Toll-like receptors (TLR), exists in vertebrates. Rock F L et al. (1998) Proc. Natl. Acad. Sci. U.S.A. 95:588-593. So far, eleven members (TLR1-TLR11) have been reported that are fundamental for the innate immune system to recognize pathogen-associated molecular patterns (PAMP) such as lipopolysaccharide, peptidoglycan, flagellin, and unmethylated bacterial CpG-DNA. Takeda K et al. (2003) Annu. Rev. Immunol. 21:335-76. Upon activation, TLR induce a signaling pathway leading to activation of transcription factors (nuclear factor kappa B (NF-κB), activator protein 1 (AP1)) and subsequent gene expression of co-stimulatory proteins and proinflammatory cytokines. Takeda K et al. (2003) Annu. Rev. Immunol. 21:335-76.

### SUMMARY OF THE INVENTION

Described herein are methods for identifying agents that affect TLR signaling. The methods of the invention can be performed as cell-free methods, i.e., without the use of cells expressing a TLR. Such methods will find use in the identification of compounds that may be useful in treating any of a variety of diseases and disorders in which immune reactivity has a role. Such conditions can include, without limitation, autoimmune diseases, inflammation and inflammatory disorders, allergy, asthma, infectious diseases, transplant rejection, and cancer.

In one aspect the invention provides a cell-free method for identifying a compound that affects TLR signaling. The method according to this aspect of the invention includes the steps of contacting an isolated polypeptide comprising a TLR extracellular domain or fragment thereof with a TLR ligand, at an acid pH in absence of a test compound, to measure a reference amount of binding between the isolated polypeptide and the TLR ligand; contacting the isolated polypeptide comprising the TLR extracellular domain or fragment thereof with the TLR ligand, at the acid pH in presence of a test compound, to measure a test amount of binding between the isolated polypeptide and the TLR ligand; and determining the test compound affects TLR signaling when the test amount of binding differs from the reference amount of binding by a defined amount.

In this and other aspects of the invention, in one embodiment the defined amount by which the test amount of binding differs from the reference amount of binding is at least 5 percent of the reference amount of binding.

In this and other aspects of the invention, in one embodiment the acid pH in absence of the test compound and the acid pH in presence of the test compound are each a pH between 4.5 and 6.9, inclusive.

In this and other aspects of the invention, in one embodiment the acid pH in absence of the test compound and the acid pH in presence of the test compound are each a pH between 5.0 and 6.9, inclusive.

In this and other aspects of the invention, in one embodiment the acid pH in absence of the test compound is selected as the acid pH in presence of the test compound.

In this and other aspects of the invention, in one embodiment the TLR ligand is a TLR ligand immobilized on a solid substrate.

In one aspect the invention provides a cell-free method for identifying a compound that affects TLR9 signaling. The method according to this aspect of the invention includes the steps of contacting an isolated polypeptide comprising a TLR9 extracellular domain or fragment thereof with a TLR9 ligand, at an acid pH in absence of a test compound, to measure a reference amount of binding between the isolated polypeptide and the TLR9 ligand; contacting the isolated polypeptide comprising the TLR9 extracellular domain or fragment thereof with the TLR9 ligand, at the acid pH in presence of a test compound, to measure a test amount of binding between the isolated polypeptide and the TLR9 ligand; and determining the test compound affects TLR9 signaling when the test amount of binding differs from the reference amount of binding by a defined amount.

In an embodiment according to this aspect of the invention the polypeptide comprising a TLR9 extracellular domain is TLR9. In an embodiment according to this aspect of the invention the polypeptide comprising a TLR9 extracellular domain is human TLR9. In an embodiment according to this aspect of the invention TLR9 ligand is CpG-DNA. In an embodiment according to this aspect of the invention the isolated polypeptide comprising a TLR9 extracellular domain or fragment thereof comprises a methyl-CpG-DNA binding domain (MBD)-like binding region.

In one aspect the invention provides a cell-free method for identifying a compound that affects TLR7 signaling. The method according to this aspect of the invention includes the steps of contacting an isolated polypeptide comprising a TLR7 extracellular domain or fragment thereof with a TLR7 ligand, at an acid pH in absence of a test compound, to measure a reference amount of binding between the isolated polypeptide and the TLR7 ligand; contacting the isolated polypeptide comprising the TLR7 extracellular domain or fragment thereof with the TLR7 ligand, at the acid pH in presence of a test compound, to measure a test amount of binding between the isolated polypeptide and the TLR7 ligand; and determining the test compound affects TLR7 signaling when the test amount of binding differs from the reference amount of binding by a defined amount.

In an embodiment according to this aspect of the invention the polypeptide comprising a TLR7 extracellular domain is TLR7. In an embodiment according to this aspect of the invention the polypeptide comprising a TLR7 extracellular domain is human TLR7.

In an embodiment according to this aspect of the invention the TLR7 ligand is RNA. In an embodiment according to this aspect of the invention the TLR7 ligand is single-stranded RNA.

In one aspect the invention provides a cell-free method for identifying a compound that affects TLR8 signaling. The method according to this aspect of the invention includes the steps of contacting an isolated polypeptide comprising a TLR8 extracellular domain or fragment thereof with a TLR8 ligand, at an acid pH in absence of a test compound, to measure a reference amount of binding between the isolated polypeptide and the TLR8 ligand; contacting the isolated polypeptide comprising the TLR8 extracellular domain or fragment thereof with the TLR8 ligand, at the acid pH in presence of a test compound, to measure a test amount of binding between the isolated polypeptide and the TLR8 ligand; and determining the test compound affects TLR8 signaling when the test amount of binding differs from the reference amount of binding by a defined amount.

In an embodiment according to this aspect of the invention the polypeptide comprising a TLR8 extracellular domain is TLR8. In an embodiment according to this aspect of the invention the polypeptide comprising a TLR8 extracellular domain is human TLR8.

In an embodiment according to this aspect of the invention the TLR8 ligand is RNA. In an embodiment according to this aspect of the invention the TLR8 ligand is single-stranded RNA.

In one aspect the invention provides a cell-free method for identifying a compound that affects TLR3 signaling. The method according to this aspect of the invention includes the steps of contacting an isolated polypeptide comprising a TLR3 extracellular domain or fragment thereof with a TLR3 ligand, at an acid pH in absence of a test compound, to measure a reference amount of binding between the isolated polypeptide and the TLR3 ligand; contacting the isolated polypeptide comprising the TLR3 extracellular domain or fragment thereof with the TLR3 ligand, at the acid pH in presence of a test compound, to measure a test amount of binding between the isolated polypeptide and the TLR3 ligand; and determining the test compound affects TLR3 signaling when the test amount of binding differs from the reference amount of binding by a defined amount.

In one embodiment according to this aspect of the invention the polypeptide comprising a TLR3 extracellular domain is TLR3. In one embodiment according to this aspect of the invention the polypeptide comprising a TLR3 extracellular domain is human TLR3.

In one embodiment according to this aspect of the invention the TLR3 ligand is RNA. In one embodiment the TLR3 ligand is double-stranded RNA.

### BRIEF DESCRIPTION OF THE DRAWINGS

FIG. 1A is an image of an SDS-PAGE gel depicting fusion proteins consisting of the extracellular domain of TLR9 or TLR2 and IgG1-Fc (mTLR9ect, mTLR2ect) purified via protein A affinity chromatography and separated by SDS-PAGE in reducing (+) and non reducing (−) conditions.

FIG. 1B is an image of an SDS-PAGE gel depicting purified proteins from FIG. 1A UV-crosslinked with ³²P-labeled CpG- or non-CpG-DNA, separated by SDS-PAGE, and autoradiographed. TLR9 and free DNA are highlighted.

FIG. 1C is a graph depicting surface plasmon resonance (SPR) biosensor analysis of TLR9-CpG-DNA interaction. Biotinylated CpG-DNA and non-CpG-DNA were immobilized on flow cells 1 and 2 of streptavidin-coated sensor chips, respectively, and sensorgrams recorded. mTLR9ect, mTLR2ect, and IgG1 were injected at pH 6.5 and 200 nM or as indicated.

FIG. 1D is a graph depicting SPR biosensor analysis of TLR9-CpG-DNA interaction. TLR9 was injected and indicated concentrations of CpG-DNA were added in the dissociation phase to examine the release of bound TLR9.

FIG. 1E is a graph depicting SPR biosensor analysis of TLR9-CpG-DNA interaction. TLR9 was subjected to analysis after preincubation with CpG- or non-CpG-DNA.

FIG. 1F is a graph depicting SPR biosensor analysis of TLR9-CpG-DNA interaction. TLR9 was injected at pH 7.4, 6.5, and 5.5. One representative experiment of at least two independent experiments is shown.

FIG. 2A is a graph depicting SPR biosensor analysis of chloroquine (CQ) on TLR9-PAMP interaction. Biotinylated CpG-DNA 1668 (SEQ ID NO:1) and non-CpG-DNA 1668GC (SEQ ID NO:3) were immobilized on streptavidin sensor chip flow cells 1 and 2, respectively. 200 nM TLR9 and indicated concentrations of chloroquine were injected and sensorgrams recorded.

FIG. 2B is a graph depicting SPR biosensor analysis of quinoquine (QC) on TLR9-PAMP interaction. Biotinylated CpG-DNA 1668 (SEQ ID NO:1) and non-CpG-DNA 1668GC (SEQ ID NO:3) were immobilized on streptavidin sensor chip flow cells 1 and 2, respectively. 200 nM TLR9 and indicated concentrations of quinoquine were injected and sensorgrams recorded.

FIG. 2C is a bar graph depicting activation of HEK 293 cells transfected with murine TLR9 and a 6-fold NF-κB luciferase reporter plasmid and stimulated with 1 μM CpG-DNA, 1 μM non-CpG-DNA, or 1 μM CpG-DNA and indicated concentrations of chloroquine (CQ) or quinacrine (QC). Activation is expressed as fold induction compared to no stimulation.

FIG. 2D is a graph depicting SPR biosensor analysis of chloroquine (CQ) on TLR2-PAMP interaction. Biotinylated Pam3Cys and the non-active analog PHC were immobilized on streptavidin sensor chip flow cells 1 and 2, respectively. 200 nM TLR2 and indicated concentrations of chloroquine were injected and sensorgrams recorded.

FIG. 2E is a graph depicting SPR biosensor analysis of quinoquine (QC) on TLR2-PAMP interaction. Biotinylated Pam3Cys and the non-active analog PHC were immobilized on streptavidin sensor chip flow cells 1 and 2, respectively. 200 nM TLR2 and indicated concentrations of quinoquine were injected and sensorgrams recorded.

FIG. 2F is a bar graph depicting activation of HEK 293 cells transfected with murine TLR2 and a 6-fold NF-κB luciferase reporter plasmid and stimulated with 1 μg/ml Pam3Cys, 1 μg/ml PHC, or 1 μg/ml Pam3Cys and indicated concentrations of chloroquine (CQ) or quinacrine (QC). Activation is expressed as fold induction compared to no stimulation.

FIG. 3 depicts a partial alignment of the MBD-domain of murine methyl-CpG-DNA binding proteins and murine TLR9. (*) marks amino acids which have been identified by mutation analysis to directly interact with methylated CpG-DNA. Amino acids (aa) shadowed in black are identical aa, whereas gray shading depicts similar aa. SEQ ID NOs are assigned as follows: MBD1, SEQ ID NO:7; MBD2, SEQ ID NO:8; MBD3, SEQ ID NO:9; MBD4, SEQ ID NO:10; MeCP2, SEQ ID NO:11; mTLR9, SEQ ID NO:12.

FIG. 3B is a graph depicting SPR biosensor analysis of wild-type and double mutated mTLR9ect (mTLR9ect-mut, D535→A and Y537→A). Proteins were injected at 200 nM on sensor chips with immobilized CpG- and non-CpG-DNA and the sensorgrams recorded.

FIG. 3C is an image of a Western blot depicting full length wild-type or mutated TLR9. HEK 293 cells were transfected with full length wild-type TLR9 (mTLR9) or mutated TLR9 (mTLR9mut, D535→A and Y537→A) and a 6-fold NF-κB luciferase reporter plasmid. An aliquot of the cells was lysed and mTLR9 detected in a western blot analysis.

FIG. 3D is a bar graph depicting activation of mTLR9- and mTLR9mut-transfected cells from FIG. 3C. Cells were stimulated with 1 μM CpG-DNA or 10 ng/ml of the TLR-independent stimulus 12-O-tetradecanoylphorbol 13-acetate (TPA) and subsequent NF-κB induction was analyzed. Activation is expressed as fold induction compared to no stimulation.

### DETAILED DESCRIPTION OF THE INVENTION

The invention is based in part on the discovery that it can be shown using surface plasmon resonance (SPR) biosensor technology that TLR9 directly interacts with CpG-DNA at the acidic pH found in endosomal/lysosomal vesicles. In addition, the invention is also based in part on the discovery that interaction between TLR9 and CpG-DNA is blocked directly by chloroquine and quinacrine. The invention is also based in part on the discovery of a region within TLR9 that shares homology to the methyl-CpG-DNA binding domain (MBD) and participates in CpG-DNA binding. Mutations of amino acids in TLR9 which are critical for DNA binding in MBD proteins strongly diminish interaction between TLR9 and CpG-DNA, and they strongly diminish CpG-DNA-driven NF-κB activation.

In one aspect the invention provides a cell-free method for identifying a compound that affects TLR signaling. The method according to this aspect of the invention includes the steps of contacting an isolated polypeptide comprising a TLR extracellular domain or fragment thereof with a TLR ligand, at an acid pH in absence of a test compound, to measure a reference amount of binding between the isolated polypeptide and the TLR ligand; contacting the isolated polypeptide comprising the TLR extracellular domain or fragment thereof with the TLR ligand, at the acid pH in presence of a test compound, to measure a test amount of binding between the isolated polypeptide and the TLR ligand; and determining the test compound affects TLR signaling when the test amount of binding differs from the reference amount of binding by a defined amount.

As used herein, the term “TLR” refers generally to any Toll-like receptor, including TLR1, TLR2, TLR3, TLR4, TLR5, TLR6, TLR7, TLR8, TLR9, TLR10, and TLR11. TLRs share certain structural features in common, including an extracellular domain, transmembrane domain, and a cytoplasmic domain, the latter known as the Toll/IL-1R (TIR) domain. Human and non-human amino acid and nucleic acid sequences for each of these TLR proteins are known and are publicly available through databases such as GenBank. Both natural and non-natural (synthetic) ligands have been described for most of these TLRs.

As used herein, an “isolated polypeptide” refers to a polypeptide that has been removed from an environment in which it is found in nature. An isolated polypeptide thus includes a polypeptide removed from a cell that expresses the polypeptide.

As used herein, a “TLR ligand” refers to a molecule that interacts with a TLR and is able to evoke signaling by the TLR under conditions that are suitable for such interaction and such signaling. In a preferred embodiment a TLR ligand refers to a molecule that interacts with an extracellular domain of a TLR. Particularly in reference to TLR7, TLR8, and TLR9, which are usually found within the endosomal/lysosomal compartment of a cell, rather than on the cell membrane or outer surface of a cell, the extracellular domain of a TLR can refer to the extracytoplasmic domain of the TLR.

A TLR ligand in one embodiment can be a TLR ligand that is found in nature, e.g., a natural ligand. For example, a natural ligand of TLR9 can be bacterial DNA. A TLR ligand in another embodiment can be a TLR ligand that is not a natural ligand. For example, a ligand for TLR7 that is not a natural ligand can be a small molecule such as imiquimod or resiquimod. As another example, a ligand for TLR9 that is not a natural ligand can be a synthetic CpG oligodeoxyribonucleotide.

As used herein, a “test compound” refers to any suitable naturally occurring, synthetic, or semi-synthetic molecule. In one embodiment the test compound is a small molecule, e.g., a synthetic organic molecule, with a molecular weight of less than about 5000 Daltons. In an embodiment the test compound is a biomolecule such as a protein, polypeptide, peptide, polynucleotide (i.e., two or more nucleotides linked together), lipid, carbohydrate, as well as derivatives thereof.

As used herein, the term “CpG-DNA” refers to a DNA molecule having a 5′ cytosine-guanine 3′ (5′-CG-3′) dinucleotide in which at least the cytosine is unmethylated and the cytosine (C) and guanine (G) nucleotides are linked through a phosphate linkage. In one embodiment the CpG-DNA is an oligonucleotide. In one embodiment the CpG-DNA includes at least one phosphate linkage that is stabilized with respect to nuclease activity, such as a phosphorothioate linkage, as compared to a phosphodiester linkage.

Part of the methods of the invention entails determining the test compound affects TLR signaling when the test amount of binding differs from the reference amount of binding by a defined amount. The defined amount by which the test amount of binding differs from the reference amount of binding can be any objectively measurable amount. In one embodiment the defined amount by which the test amount of binding differs from the reference amount of binding is at least 5 percent of the reference amount of binding. In various embodiments, the defined amount by which the test amount of binding differs from the reference amount of binding is at least 10 percent, at least 20 percent, at least 30 percent, at least 40 percent, at least 50 percent, at least 60 percent, at least 70 percent, at least 80 percent, or at least 90 percent of the reference amount of binding. In one embodiment the test amount of binding will be less than the reference amount of binding.

The test and reference amounts of binding between an isolated polypeptide comprising a TLR extracellular domain, or a fragment thereof, and a TLR ligand can be measured using any suitable method. In one embodiment, the amount of binding is measured using SPR biosensor technology.

TLR9 recognizes unmethylated bacterial and synthetic CpG-DNA and activates immune cells. Bauer S et al. (2001) Proc. Natl. Acad. Sci. U.S.A. 98:9237-9242; Hemmi H et al. (2000) Nature 408:740-745. The stimulatory effect of bacterial and synthetic CpG-DNA is due to the presence of unmethylated CpG dinucleotides in a particular base context named CpG-motif. Krieg A M et al. (1995) Nature 374:546-549. Human and murine immune cells differ in their preference for the core CpG-motif. Mouse cells respond better to CpG-DNA containing the core sequence GACGTT, whereas human cells prefer CpG-motifs containing more than one CG and the core sequence GTCGTT. Bauer S et al. (2001) Proc. Natl. Acad. Sci. U.S.A. 98:9237-9242. Receptor-mediated endocytosis of CpG-DNA, endosomal acidification (maturation), and CpG-DNA recognition in endosomal/lysosomal vesicles by TLR9 are believed to be essential steps for cellular activation. Hacker H et al. (1998) EMBO J. 17:6230-6240; Yi A K et al. (1998) J. Immunol. 160:4755-4761; Ahmad-Nejad P et al. (2002) Eur. J. Immunol. 32:1958-1968. Compounds interfering with endosomal acidification, such as chloroquine and bafilomycin A1, inhibit signaling. Hacker H et al. (1998) EMBO J. 17:6230-6240; Macfarlane D E et al. (1998) J. Immunol. 160:1122-1131. Interestingly, chloroquine and the analog quinacrine serve as therapeutics for autoimmune diseases like rheumatoid arthritis and systemic lupus erythematosus (SLE), but the mechanism of their action is unknown. Furst D E et al. (1999) Arthritis Rheum. 42:357-365; The Canadian Hydroxychloroquine Study Group. (1991) A randomized study of the effect of withdrawing hydroxychloroquine sulfate in systemic lupus erythematosus. N. Engl. J. Med. 324:150-154.

DNA-protein interaction is mediated by certain protein binding motifs such as leucine-zipper, helix-turn-helix, or the zinc-finger motif. Struhl K. (1989) Trends Biochem. Sci. 14:137-140. A recently discovered family of methylated CpG-DNA binding proteins (MBD1-4), which has important functions in DNA-methylation-dependent gene silencing and chromatin remodeling, utilizes a different DNA-binding motif termed the MBD domain. Hendrich B et al. (1998) Mol. Cell Biol. 18:6538-6547. This domain mediates the interaction with double-stranded methylated CpG-DNA. Hendrich B et al. (1998) Mol. Cell Biol. 18:6538-6547; Fujita N et al. (2000) Mol. Cell Biol. 20:5107-5118.

TLR7 has been reported to recognize certain synthetic compounds, including imidazoquinolines, loxoribine, and bropirimine, as well as certain RNA molecules. See, for example, commonly owned U.S. Pat. Application Publication 2003/0232074, and Heil F et al. (2004) Science 303:1526-1529. In particular, TLR7 is believed to signal in response to G,U-containing RNA, with certain sequence specificity. The RNA can be single-stranded or at least partially double-stranded.

TLR8 has been reported to recognize certain synthetic compounds, including imidazoquinolines as well as certain RNA molecules. See, for example, commonly owned U.S. Pat. Application Publication 2003/0232074, and Heil F et al. (2004) Science 303:1526-1529. In particular, TLR8 is believed to signal in response to G,U-containing RNA, with certain sequence specificity. The RNA can be single-stranded or at least partially double-stranded.

TLR3 has been reported to recognize double-stranded RNA. See, for example, Alexopoulou L et al. (2001) Nature 413:732-738.

The present invention is further illustrated in the following examples, which are not intended to be limiting in any way.

### EXAMPLES

The following examples demonstrate that the extracellular domain of TLR9 binds directly to CpG-DNA, whereas TLR2 does not. Using SPR biosensor technology, it was shown that TLR9-CpG-DNA interaction is pH-dependent and occurs at acidic pH found in endosomes and lysosomes (pH 6.5 to 5.0). Furthermore, chloroquine and quinacrine, therapeutics for autoimmune diseases like rheumatoid arthritis (RA) and systemic lupus erythematosus (SLE) were found to directly block TLR9-CpG-DNA interaction but not TLR2-Pam3Cys binding. A putative CpG-DNA binding region in TLR9 that is homologous to the CpG-DNA binding domain described for methyl-CpG-binding proteins (MBD) was found to participate in TLR9-CpG-DNA interaction. Amino acid substitution to this region abrogated CpG-DNA binding and led to loss in NF-κB activation. The results described below provide insight into the molecular basis of TLR-agonist interaction and also shed light on a mechanism for chloroquine/quinacrine interference with TLR9-dependent activation of self-reactive B cells in autoimmune diseases.

### Materials and Methods

Cells and reagents. Human embryonic kidney (HEK) 293 cells were obtained from American Type Culture Collection (ATCC, Manassas, Va.) and cultivated in Dulbecco's modified Eagle's medium (PAN, Aidenbach, Germany) supplemented with 7.5% fetal calf serum (FCS). Human IgG, 12-O-tetradecanoylphorbol 13-acetate (TPA), chloroquine, quinacrine, and bafilomycin A1 were obtained from Sigma-Aldrich (Taufkirchen, Germany) or Calbiochem (San Diego, USA), respectively. CpG-DNA 1668 (5′-TCCATGACGTTCCTGATGCT-3′; SEQ ID NO:1) or 2006 (5′-TCGTCGTTTTGTCGTTTTGTCGTT-3′; SEQ ID NO:2) and non-CpG-DNA 1668GC (5′-TCCATGAGCTTCCTGATGCT-3′; SEQ ID NO:3) or 2006GC (5′-TGCTGCTTTTGTGCTTTTGTGCTT-3′; SEQ ID NO:4) were synthesized by MWG Biotech (Ebersberg, Germany) as phosphodiester with or without a 3′ biotin modification and in a phosphorothioate-protected form without any additional modification by TIB BIOMOL (Berlin, Germany), respectively. Pam3CysK4 (S-(2,3-bis(palmitoyloxy)-(2-RS)-propyl)-N-palmitoyl-(R)-Cys-(S)-Ser-(S)-Lys(4)) and PHCK4 (N-Palmitoyl-S-(1,2-dicarboxyhexadecyl)ethyl-Cys-(S)-Ser-(S)-Lys(4)) were purchased in a biotinylated form from EMC microcollections GmbH (Tuebingen, Germany).

Protein. Recombinant fusion proteins consisting of the extracellular domain of Toll-like receptors mTLR9ect (aa 1-816), mTLR9ect-mut (see below) (aa 1-816), hTLR9ect (aa 1-815) and mTLR2ect (aa 1-587) fused to human IgG1-Fc were constructed by amplifying the corresponding extracellular domain and ligating the fragment in frame into a pcDNA3.1(−) (Invitrogen, Netherlands) vector containing the coding sequence for human IgG1-Fc. Fusion proteins were stably expressed in HEK 293 cells and purified from cell lysates by protein A affinity chromatography.

Mutation. For mutation of wild-type mTLR9 amino acids D535 and Y537 to two alanines, the primers 5′-CATAACAAACTGGCCTTGGCCCACTGGAAATC-3′ (SEQ ID NO:5) and 5′-GATTTCCAGTGGGCCAAGGCCAGTTTGTTATG-3′ (SEQ ID NO:6) were used to generate mTLR9-mut. A site-specific-mutagenesis kit from Stratagene (Amsterdam, Netherlands) was applied according to the manufacturer's protocol. All PCR fragments were sequenced and found error-free.

NF-κB luciferase assay. For monitoring transient NF-κB activation, 3×10⁶ HEK 293 cells were electroporated at 200 volt and 950 μF with 20 ng of a NF-κB luciferase reporter plasmid (kindly provided by Patrick Baeuerle, Munich, Germany) and 1 μg mTLR2 (kindly provided by Tularik, Inc., South San Francisco, USA), mTLR9, or mTLR9-mut expression plasmid. Cells were seeded at 10⁵ cells per well and after overnight culture stimulated with 1 μM phosphorothioated CpG-DNA 1668 (SEQ ID NO:1), 1 μM non-CpG-DNA 1668GC (SEQ ID NO:3), 1 μg/ml Pam3CysK4 or 1 μg/ml PHCK4 for further 8 hours. In some experiments chloroquine, quinacrine, or bafilomycin A1 were added 15 min prior to stimulation at indicated concentrations. Stimulated cells were lysed using reporter lysis buffer (Promega, Mannheim, Germany) and lysates were assayed for luciferase activity using a Berthold luminometer (Wildbad, Germany) according to the manufacturer's instruction.

Western blot. Transfected HEK 293 cells were lysed in lysis buffer containing 25 mM HEPES, 150 mM NaCl, 1% octylglycopyranoside. Lysates were boiled in SDS sample buffer, sonicated, resolved by 10% SDS-PAGE, and blotted onto a polyvinylidene fluoride (PVDF) hydrophobic membrane (Immobilon-P, Millipore, Germany). Membranes were blocked in 5% skim milk solution, probed with the murine TLR9-specific antibody 5G5 (HBT, Netherlands), a polyclonal peroxidase-conjugated goat anti mouse IgG (1:5000) (Dianova, Germany), and subsequently visualized using the chemiluminescence West Dura detection system (Pierce, Perbio Science, Germany).

SPR biosensor analysis. Real-time binding of TLR9 or TLR2 was measured by surface plasmon resonance biosensor technology using the BiaCore X system (Uppsala, Sweden). For analysis of TLR9 and TLR2 interaction, biotinylated CpG-DNA and non-CpG-DNA, or biotinylated Pam3CysK4 and PHCK4, respectively, were loaded in running buffer (50 mM MES, 150 mM NaCl, 1 mM MgCl₂ at pH 6.5) on SA chips precoated with streptavidin (Biacore AB, Uppsala, Sweden). Non-CpG-DNA or PHCK4 (Wiesmuller K H et al. (1989) Vaccine 7:29-33) served as reference for CpG-DNA and Pam3CysK4 interaction (structure or sequence in Table 1). Displayed figures show subtracted binding curves between flow cell 2 (CpG-DNA or Pam3CysK4) and flow cell 1 (non-CpG-DNA or PHCK4), respectively. TLR proteins were introduced at 200 nM or as indicated in 40 μl running buffer at a flow rate of 10 μl/min. Binding was measured at 25° C. for 750 s (delay time 300 s). For some experiments TLR proteins were mixed with chloroquine, quinacrine, or bafilomycin A1 (adjusted pH), free non-biotinylated phosphodiester CpG-DNA or non-CpG-DNA prior to injection. pH-dependent TLR9-CpG-DNA interaction was analyzed by injecting TLR9 at different pH onto an equilibrated sensor chip. Sensorgrams were recorded and kinetic data were calculated by the BiaCore Evaluation program (Biacore, version 3.0.1). Regeneration of the chip was performed by injection of 10 μl 50 mM NaOH, 1 M NaCl and extensive re-equilibration.

### Example 1

### TLR9 Binds Directly to CpG-DNA in a pH-Dependent Manner

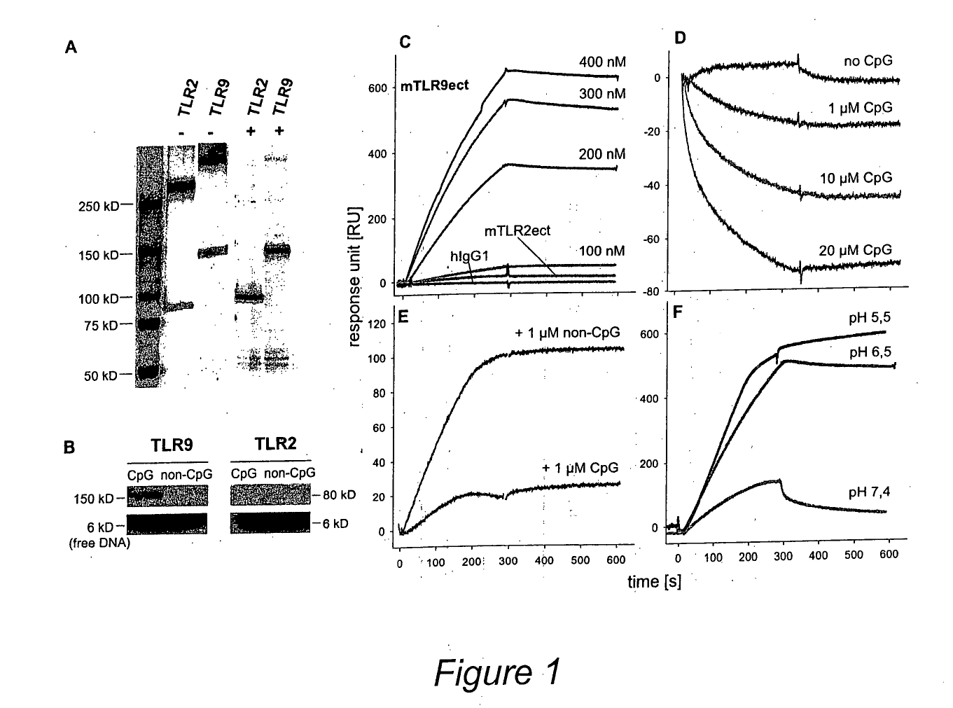

Genetic complementation experiments suggest interaction of TLR9 and CpG-DNA, however direct interaction has not been demonstrated. Bauer S et al. (2001) Proc. Natl. Acad. Sci. U.S.A. 98:9237-9242. To assess binding of TLR9 to CpG-DNA, a recombinant fusion protein consisting of the extracellular domain of murine TLR9 and human IgG 1-Fc (mTLR9ect) was constructed. The extracellular domain of murine TLR2 also fused to IgG1-Fc (mTLR2ect) served as control. Proteins were expressed in HEK 293 cells and purified via protein A affinity chromatography (FIG. 1A). Under denaturing and non-reducing conditions mTLR9ect and mTLR2ect are approximately 10% in a monomeric and 90% in a dimeric form. Dimerization of the proteins is probably mediated by the IgG 1-Fc fusion partner which forms disulfide bonds (FIG. 1A). Initially, binding of mTLR9ect to radiolabeled DNA was determined by UV-crosslinking and subsequent SDS-PAGE. As shown in FIG. 1B, mTLR9ect bound to CpG-DNA 1668 (SEQ ID NO:1) and formed a complex at the expected size of 150 kDa. In contrast, non-CpG-DNA 1668GC (SEQ ID NO:3) bound only weakly and mTLR2ect did not interact with DNA.

For detailed analysis of TLR9-CpG-DNA interaction, surface plasmon resonance (SPR) biosensor based technology was used with CpG- and non-CpG-DNA immobilized onto separate flow cells of a streptavidin-coated chip. Under these settings non-CpG-DNA served as reference for specific binding (see Methods). Binding of mTLR9ect to DNA was CpG-sequence-specific and increased in a dose-dependent manner. In contrast, mTLR2ect and human IgG1 showed no binding to DNA (FIG. 1C). Using the 1:1 binding model of the BioEvaluation software as fitting algorithm and on the basis of four different mTLR9ect concentrations ranging from 50 to 300 nM, a dissociation constant (K_{d}) of 200 nM was obtained with low chi-squared residuals (4,76). The calculated K_{d} is similar to that of soluble Toll interacting with Spaetzle (82 nM). Weber AN et al. (2003) Nat. Immunol. 4:794-800. Furthermore the concentration of CpG-DNA 1668 (SEQ ID NO:1) for half-maximal activation of murine TLR9 has been recently calculated as 70 nM which correlates well with the K_{D} obtained here. Bauer S et al. (2001) Proc. Natl. Acad. Sci. U.S.A. 98:9237-9242. Similar binding data were obtained for human TLR9ect which specifically interacted with the CpG-DNA 2006 (SEQ ID NO:2) (non-CpG-DNA (SEQ ID NO:4) served as control) although the interaction was weaker compared to mTLR9-CpGDNA binding. This difference in affinity correlates with the observed species-specific variance in CpG-motif recognition, as well as activation potential. Bauer S et al. (2001) Proc. Natl. Acad. Sci. U.S.A. 98:9237-9242.

Specificity of the TLR9-CpG-DNA interaction was further assessed by competition experiments. Free injected CpG-DNA competed with immobilized CpG-DNA and dose dependently released bound mTLR9ect (FIG. 1D). In contrast, non-CpG-DNA did not lead to the release of bound mTLR9ect. Furthermore, pre-incubation of mTLR9ect with CpG-DNA prior to SPR biosensor analysis abolished binding to chip-immobilized CpG-DNA, whereas non-CpG-DNA had no inhibitory effect on CpG-DNA interaction (FIG. 1E).

Since endosomal acidification (maturation) is a prerequisite for CpG-DNA activity (Hacker H et al. (1998) EMBO J. 17:6230-6240; Yi A K et al. (1998) J. Immunol. 160:4755-4761), the pH dependence of interaction between TLR9 and CpG-DNA was examined. At physiological pH (pH 7.4) TLR9 binding to CpG-DNA was weak (FIG. 1F) and dissociation occurred fairly rapidly. Lowering the pH to 6.5 or 5.5 led to a strong TLR9-CpG-DNA binding (FIG. 1F) with weak dissociation. The high affinity interaction of TLR9 and CpG-DNA at acidic pH found in endosomes and lysosomes (pH 6.5 to 4.5 (Mellman I et al. (1986) Annu. Rev. Biochem. 55:663-700)) supports the model that TLR9-driven signaling is initiated from endosomal/lysosomal vesicles after CpG-DNA binding.

Utilizing SPR biosensor technology, results of these experiments show for the first time direct binding of TLR9 and CpG-DNA and further extend previous findings of direct TLR-PAMP interaction. da Silva C J et al. (2001) J. Biol. Chem. 276:21129-21135; Murakami S et al. (2002) J. Biol. Chem. 277:6830-6837; Iwaki D et al. (2002) J. Biol. Chem. 277:24315-24320.

### Example 2

### Binding of TLR9 and CpG-DNA is Inhibited by Chloroquine and Quinacrine

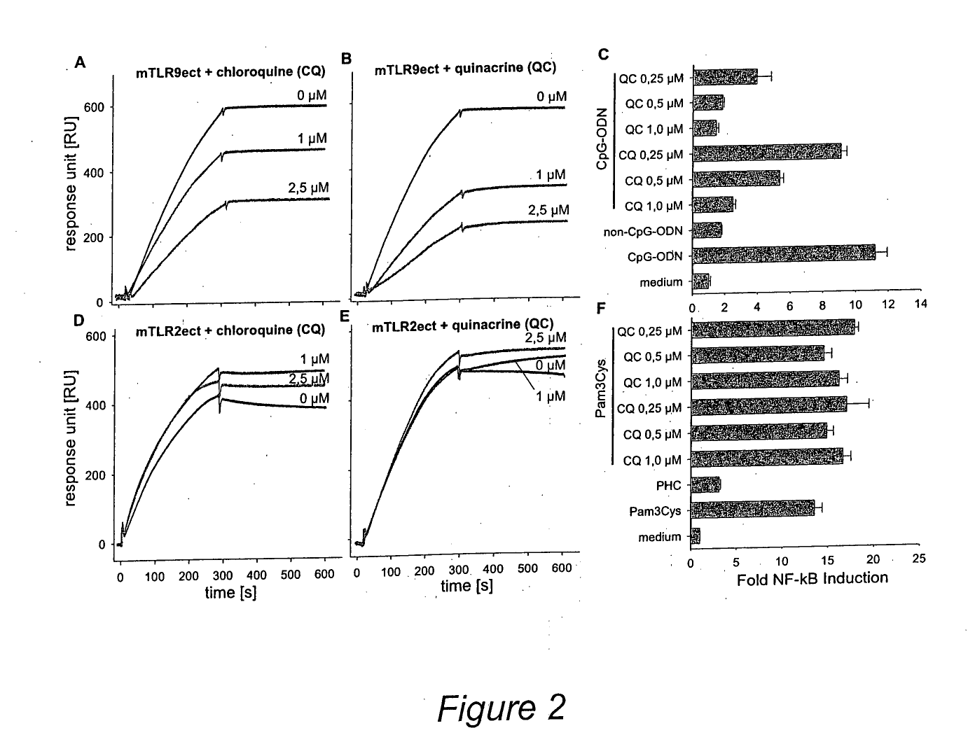

CpG-DNA driven signaling via TLR9 requires acidification and maturation of endosomes. Hacker H et al. (1998) EMBO J. 17:6230-6240; Yi A K et al. (1998) J. Immunol. 160:4755-4761; Ahmad-Nejad P et al. (2002) Eur. J. Immunol. 32:1958-1968. CpG-DNA signaling is efficiently blocked by dominant negative Rab5, bafilomycin A1, chloroquine, and quinacrine, which interfere with endosomal trafficking or acidification, respectively. Hacker H et al. (1998) EMBO J. 17:6230-6240; Yi A K et al. (1998) J. Immunol. 160:4755-4761; Macfarlane D E et al. (1998) J. Immunol. 160:1122-1131. At high concentrations chloroquine or quinacrine are weak bases that can partition into endosomes and neutralize the pH. Since both substances block the activity of immunostimulatory CpG-DNA at concentrations much below those needed for pH interference, a different mechanism was envisioned for their action. Macfarlane D E et al. (1998) J. Immunol. 160:1122-1131. Supported by the observation that chloroquine analogs without buffering capacity block CpG-DNA driven signaling, these findings suggest that chloroquine and related compounds interfere with TLR9-CpG-DNA interaction (G. Lipford, unpublished observation). Manzel L et al. (1999) J. Pharmacol. Exp. Ther. 291:1337-1347. In fact, chloroquine and quinacrine dose-dependently inhibit binding of TLR9 to CpG-DNA as well as TLR9-driven NF-κB activation in TLR9-transfected HEK 293 cells (FIG. 2A-C). Quinacrine is more potent in blocking TLR9-CpG-DNA interaction and cellular activation, consistent with previously reported data. Macfarlane D E et al. (1998) J. Immunol. 160:1122-1131. In contrast, bafilomycin A1, a specific inhibitor of the V-type ATPase which is responsible for acidification of endosomes and lysosomes (Yoshimori T et al. (1991) J. Biol. Chem. 266:17707-17712), inhibited cell activation but did not influence TLR9-CpG-DNA interaction. Specificity of chloroquine and quinacrine action on TLR9-CpG-DNA interaction was further assessed by testing their effect on a different TLR-PAMP interaction. Since the synthetic lipopeptide Pam3Cys stimulates TLR2 (Aliprantis A O et al. (1999) Science 285:736-739), a SPR biosensor based binding assay utilizing immobilized Pam3Cys and soluble TLR2 (mTLR2ect) was established. PHC, a non-active analog of PAM3Cys (Wiesmuller K H et al. (1989) Vaccine 7:29-33), was used as reference for TLR2 interaction (see Methods and Table 1). Binding of TLR2 to Pam3Cys was specific and allowed to test the effect of chloroquine and quinacrine on this interaction. In fact, both substances inhibited neither TLR2-Pam3Cys binding nor Pam3Cys-driven cellular activation, supporting their specific effect on TLR9-CpG-DNA interaction (FIG. 2D-F).

Interestingly, chloroquine and quinacrine serve as a therapeutics for autoimmune diseases like rheumatoid arthritis and systemic lupus erythematosus (SLE) which are characterized by autoantibodies against immunoglobulins, DNA and nuclear fractions. Furst D E et al. (1999) Arthritis Rheum. 42:357-365; The Canadian Hydroxychloroquine Study Group. (1991) A randomized study of the effect of withdrawing hydroxychloroquine sulfate in systemic lupus erythematosus. N. Engl. J. Med. 324:150-154. The mechanism of chloroquine action in autoimmune diseases is unknown, but recent data in a murine animal model for SLE and rheumatoid arthritis (MRL/lpr mice) suggest that its beneficial effect is due to blocking the TLR (presumably TLR9)-dependent and chromatin-antibody complex-induced stimulation of self-reactive B cells. Leadbetter E A et al. (2002) Nature 416:603-607. Here we provide mechanistic data that the therapeutic effect of chloroquine in autoimmune diseases is not due to its buffering capacity, but in fact can be attributed to its interference with TLR9-CpG-DNA binding.

### Example 3

### A Putative DNA Binding Region Mediates CpG-DNA Interaction

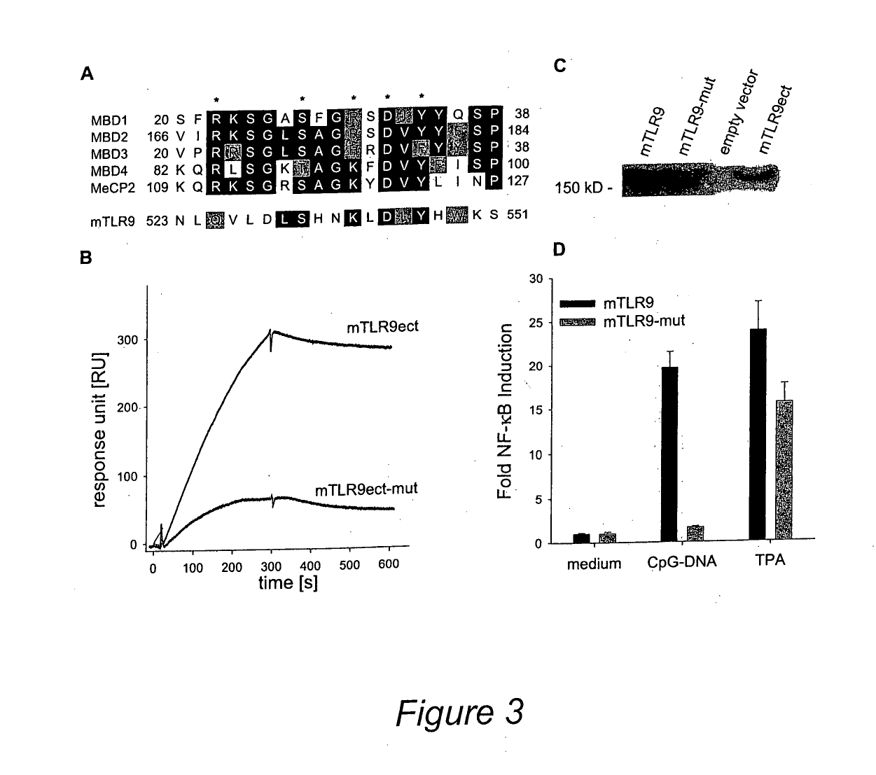

The MBD domain of methylated CpG-DNA binding proteins (MBD1-4) binds double-stranded methylated CpG-DNA. The recognition of each strand of the DNA is mediated by a loop L1/β3 structure (amino acid aa 20-37 of MBD-1) and a short loop L2/α-helical fold (aa 44-55), respectively. Ohki I et al. (2001) Cell 105:487-497. Sequence comparison of MBD proteins and TLR9 revealed a stretch of homology in the loop L1/β3 region of the MBD domain (FIG. 3A). In MBD proteins certain amino acids have been identified as direct contact points with DNA (FIG. 3A). Mutation analysis demonstrated that replacement of D32 and Y34 with alanines abolished MBD-1 mediated DNA binding. Fujita N et al. (2000). Mol. Cell Biol. 20:5107-5118; Ohki I et al. (2001) Cell 105:487-497. A double mutant of mTLR9ect (mTLR9ect-mut) was generated with alanines replacing D535 and Y537. In fact, purified mTLR9ect-mut bound only weakly to CpG-DNA when compared to wild-type mTLR9ect (FIG. 3B). Furthermore, HEK 293 cells transfected with the mutated full length mTLR9 did not respond to CpG-DNA, although the protein was expressed at similar levels to wild-type mTLR9 (FIG. 3C, D). Together these data suggest that the region containing the D535 and Y537 is involved in DNA binding, however direct interaction can not be concluded from this result. It is possible that the mutations change the folding of neighboring LRR which are involved in direct interaction with CpG-DNA. A co-crystal of TLR9 with CpG-DNA and the identification of its three-dimensional structure will elucidate the actual binding site.

Taken together this data demonstrates the direct interaction of TLR9 and CpG-DNA. Strong interaction occurs at acidic pH which exists in endosomes or lysosomes (pH 6.5 to 4.5). Our finding supports the view that CpG-DNA is transported into endosomal/lysosomal vesicles to encounter TLR9 for activation of the signaling cascade. The TLR9-CpG-DNA interaction is blocked by chloroquine and quinacrine, therapeutics in autoimmune diseases. The development of chloroquine analogs with optimized inhibition of TLR9-CpG-DNA interaction might lead to more useful anti-inflammatory drugs in autoimmune diseases in the future.

TABLE 1
Ligands and inhibitors used for SPR biosensor analysis of TLR-PAMP interaction.

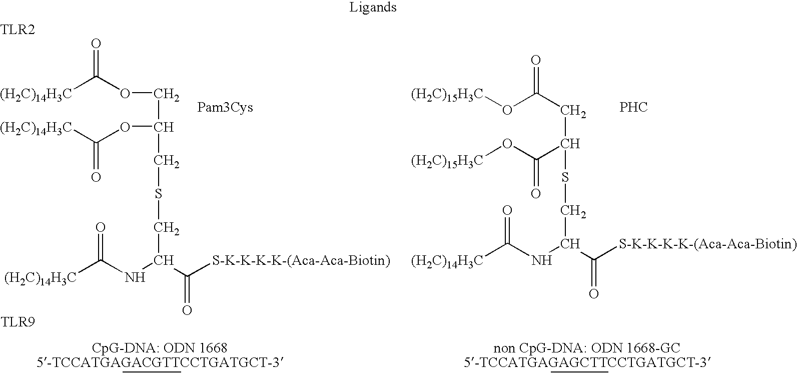

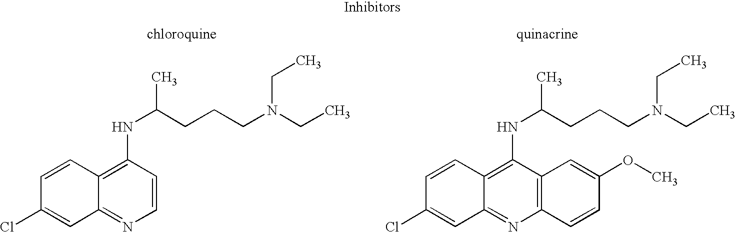

### EQUIVALENTS

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

All patents, patent applications, and references identified or cited herein are incorporated in their entirety herein by reference.

1. A cell-free method for identifying a compound that affects TLR signaling, the method comprising:
contacting an isolated polypeptide comprising a TLR extracellular domain or fragment thereof with a TLR ligand, at an acid pH in absence of a test compound, to measure a reference amount of binding between the isolated polypeptide and the TLR ligand;
contacting the isolated polypeptide comprising the TLR extracellular domain or fragment thereof with the TLR ligand, at the acid pH in presence of a test compound, to measure a test amount of binding between the isolated polypeptide and the TLR ligand; and
determining the test compound affects TLR signaling when the test amount of binding differs from the reference amount of binding by a defined amount.

2. The method of claim 1, wherein the polypeptide comprising a TLR extracellular domain is a TLR.

3. The method of claim 1, wherein the polypeptide comprising a TLR extracellular domain is a human TLR.

4. The method of claim 1, wherein the defined amount is at least 5 percent of the reference amount of binding.

5. The method of claim 1, wherein the acid pH in absence of the test compound and the acid pH in presence of the test compound are each a pH between 4.5 and 6.9, inclusive.

6. The method of claim 1, wherein the acid pH in absence of the test compound and the acid pH in presence of the test compound are each a pH between 5.0 and 6.9, inclusive.

7. The method of claim 1, wherein the acid pH in absence of the test compound is selected as the acid pH in presence of the test compound.

8. The method of claim 1, wherein the TLR ligand is a TLR ligand immobilized on a solid substrate.

9. A cell-free method for identifying a compound that affects TLR9 signaling, the method comprising:
contacting an isolated polypeptide comprising a TLR9 extracellular domain or fragment thereof with a TLR9 ligand, at an acid pH in absence of a test compound, to measure a reference amount of binding between the isolated polypeptide and the TLR9 ligand;
contacting the isolated polypeptide comprising the TLR9 extracellular domain or fragment thereof with the TLR9 ligand, at the acid pH in presence of a test compound, to measure a test amount of binding between the isolated polypeptide and the TLR9 ligand; and
determining the test compound affects TLR9 signaling when the test amount of binding differs from the reference amount of binding by a defined amount.

10-18. (canceled)

19. A cell-free method for identifying a compound that affects TLR7 signaling, the method comprising:
contacting an isolated polypeptide comprising a TLR7 extracellular domain or fragment thereof with a TLR7 ligand, at an acid pH in absence of a test compound, to measure a reference amount of binding between the isolated polypeptide and the TLR7 ligand;
contacting the isolated polypeptide comprising the TLR7 extracellular domain or fragment thereof with the TLR7 ligand, at the acid pH in presence of a test compound, to measure a test amount of binding between the isolated polypeptide and the TLR7 ligand; and
determining the test compound affects TLR7 signaling when the test amount of binding differs from the reference amount of binding by a defined amount.

20-28. (canceled)

29. A cell-free method for identifying a compound that affects TLR8 signaling, the method comprising:
contacting an isolated polypeptide comprising a TLR8 extracellular domain or fragment thereof with a TLR8 ligand, at an acid pH in absence of a test compound, to measure a reference amount of binding between the isolated polypeptide and the TLR8 ligand;
contacting the isolated polypeptide comprising the TLR8 extracellular domain or fragment thereof with the TLR8 ligand, at the acid pH in presence of a test compound, to measure a test amount of binding between the isolated polypeptide and the TLR8 ligand; and
determining the test compound affects TLR8 signaling when the test amount of binding differs from the reference amount of binding by a defined amount.

30-38. (canceled)

39. A cell-free method for identifying a compound that affects TLR3 signaling, the method comprising:
contacting an isolated polypeptide comprising a TLR3 extracellular domain or fragment thereof with a TLR3 ligand, at an acid pH in absence of a test compound, to measure a reference amount of binding between the isolated polypeptide and the TLR3 ligand;
contacting the isolated polypeptide comprising the TLR3 extracellular domain or fragment thereof with the TLR3 ligand, at the acid pH in presence of a test compound, to measure a test amount of binding between the isolated polypeptide and the TLR3 ligand; and
determining the test compound affects TLR3 signaling when the test amount of binding differs from the reference amount of binding by a defined amount.

40-48. (canceled)


SEQUENCE LISTING (12 sequences)

SEQ ID NO: 1 - DNA, 20 bp, Artificial sequence
  Synthetic oligonucleotide
tccatgacgttcctgatgct

SEQ ID NO: 2 - DNA, 24 bp, Artificial sequence
  Synthetic oligonucleotide
tcgtcgttttgtcgttttgtcgtt

SEQ ID NO: 3 - DNA, 20 bp, Artificial sequence
  Synthetic oligonucleotide
tccatgagcttcctgatgct

SEQ ID NO: 4 - DNA, 24 bp, Artificial sequence
  Synthetic oligonucleotide
tgctgcttttgtgcttttgtgctt

SEQ ID NO: 5 - DNA, 32 bp, Artificial sequence
  Synthetic oligonucleotide
cataacaaactggccttggcccactggaaatc

SEQ ID NO: 6 - DNA, 32 bp, Artificial sequence
  Synthetic oligonucleotide
gatttccagtgggccaaggccagtttgttatg

SEQ ID NO: 7 - protein, 19 aa, Mus musculus
SFRKSGASFGRSDIYYQSP

SEQ ID NO: 8 - protein, 19 aa, Mus musculus
VIRKSGLSAGRSDVYYFSP

SEQ ID NO: 9 - protein, 19 aa, Mus musculus
VPRRSGLSAGHRDVFYYSP

SEQ ID NO: 10 - protein, 19 aa, Mus musculus
KQRLSGKTAGKFDVYFISP

SEQ ID NO: 11 - protein, 19 aa, Mus musculus
KQRKSGRSAGKYDVYLINP

SEQ ID NO: 12 - protein, 19 aa, Mus musculus
NLQVLDLSHNKLDLYHWKS